(19) United States
(12) Patent Application Publication
(10) Pub. No.: US 2026/0294797 A1
(43) Pub. Date: Oct. 1, 2026

(54) OPHTHALMIC SOLUTIONS FOR CANINE, FELINE & EQUINE GLAUCOMA AND OHT

(71) Applicant: Cayman Chemical Company Inc., Ann Arbor, MI (US)

(72) Inventors: Stephen Douglas Barrett, Hartland, MI (US); Mitchell DE LONG, Chapel Hill, NC (US)

(21) Appl. No.: 19/629,746

(22) Filed: Mar. 26, 2026

Related U.S. Application Data
(60) Provisional application No. 63/778,735, filed on Mar. 27, 2025.

(51) Int. Cl. A61K 9/00; A61K 9/08; A61K 31/381; A61K 31/444; A61K 31/472; A61K 31/5575; A61K 31/559; A61K 45/06; A61K 47/02; A61K 47/14; A61K 47/18; A61K 47/26; A61K 47/44

(52) U.S. Cl. CPC A61K 9/0048; A61K 9/08; A61K 31/381; A61K 31/444; A61K 31/472; A61K 31/5575; A61K 31/559; A61K 45/06; A61K 47/02; A61K 47/14; A61K 47/186; A61K 47/26; A61K 47/44

(57) ABSTRACT

The present invention provides an ophthalmic composition comprising ING or KMN-159 and about 0.01% weight/volume to about 10% weight/volume of a tonicity agent.

### CROSS REFERENCE TO RELATED APPLICATIONS

This application claims the benefit of priority under 35 U.S.C. § 119 (e) of U.S. Provisional Application No. 63/778,735, filed Mar. 27, 2025, the disclosure of which is incorporated by reference herein in its entirety.

### BACKGROUND

Despite decades of research in the treatment of human glaucoma and ocular hypotension (OHT), little effort has been put into discovery research for the treatment of domestic animals, including pets and horses. The veterinarian is often left to pick among drugs designed and intended for humans, often with inferior and unsatisfactory results, despite the fact some breeds of dogs, in particular, suffer from closed-angle glaucoma at a much higher rate than do humans, even approaching 5% of the population of certain breeds. With the advent of pandemics and the increase in pet ownership that accompanies them, large numbers of domestic animals are now in danger of losing their sight due to the lack of research in this area. Ophthalmic compositions are clearly needed that are specifically intended for, in particular, dogs, cats and horses.

### SUMMARY

In one aspect, the present disclosure provides an ophthalmic composition for dogs, cats and horses, comprising: a selective EP2 or EP4 agent and a carrier for the treatment of canine, feline & equine glaucoma and ocular hypertension (OHT).

In a second aspect, the present disclosure provides a method for treating glaucoma, ocular hypertension (OHT) and other ophthalmic diseases in an animal in need thereof, the method comprising administering a therapeutically effective amount of a composition comprising at least one active agent selected from: isopropyl N-(6-(((4-(1H-pyrazol-1-yl)benzyl) (3-pyridinylsulfonyl)amino)methyl)-2-pyridinyl)glycinate (ING) and (7-((R)-3,3-difluoro-5-((3S,4S,E)-3-hydroxy-4-methylnon-1-en-6-yn-1-yl)-2-oxopyrrolidin-1-yl) heptanoic acid (KMN-159) including its free acid, pharmaceutically acceptable salts, its lower alkyl esters, or diastereomers thereof; and a tonicity agent.

### DETAILED DESCRIPTION

It has surprisingly been discovered that compounds (also referred to as agents) that selectively activate the equine, canine and feline EP2 & EP4 transmembrane receptors are useful for the treatment of glaucoma and OHT. It has been found that, particularly in cats, compounds that activate only the Prostaglandin F Receptor (FP receptor) are inferior treatment options. In an aspect, the present invention provides an ophthalmic composition comprising one or more selective EP2 and/or EP4 agents from about 0.0001% weight/volume to about 0.05% weight/volume of a solution; and about 0.0001% weight/volume to about 1% weight/volume of one or more selective EP2 and/or EP4 agents. In another aspect, the present invention provides an ophthalmic composition comprising one or more selective EP2/EP4 agents from about 0.001% weight/volume to about 5.0% weight/volume of a solution; and about 0.0001% weight/volume to about 1% weight/volume of a kinase inhibitor. In another aspect, the present invention provides an ophthalmic composition comprising one or more of the specific selective EP2 and/or EP4 agents isopropyl N-(6-(((4-(1H-pyrazol-1-yl)benzyl) (3-pyridinylsulfonyl)amino)methyl)-2-pyridinyl) glycinate (ING) and KMN-159 (7-((R)-3,3-difluoro-5-((3S,4S,E)-3-hydroxy-4-methylnon-1-en-6-yn-1-yl)-2-oxopyrrolidin-1-yl) heptanoic acid). In another aspect, the present invention provides a combination of ING and a beta blocker.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrases “at least one of A, B, . . . and N” or “at least one of A, B, . . . N, or combinations thereof” are defined in the broadest sense to mean one or more elements selected from the group comprising A, B, . . . and N, that is to say, any combination of one or more of the elements A, B, . . . or N including any one element alone or in combination with one or more of the other elements which may also include, in combination, additional elements not listed.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

For the purposes of the present invention, the phrase “EP2 agent” and/or the phrase “EP4 agent” refer to small organic molecules that bind to and selectively activate the G-coupled 7-transmembrane receptors of those names. Other receptors of the EP family of receptors include the EP1 and EP3 receptors. Closely related receptors not in the EP family include DP1, DP2, FP, IP, and thromboxane (TP) receptors. Selectively means that the binding of the compound under standard conditions yields an EC50 that is at least 5-fold better for the respective desired receptor than the other EP and closely related receptors described herein. Preferably, compounds are 10-fold selective, particularly when compared to their ability to activate the thromboxane receptor(s).

### Compositions

In an aspect, the present invention provides an ophthalmic composition comprising ING and a buffer. ING may be present in an amount of about 0.0001% w/v to about 0.06% w/v. Suitably, ING may be present in an amount of about 0.001% w/v to about 0.03%. When present together, the molar ratio of ING to KMN-159 shall be no more than about 100:1 and no less than about 1:100. For the purposes of this invention, the phrase “ING” shall include the free acid, pharmaceutically acceptable salts, the methyl, ethyl, isopropyl and isobutyl esters of the parent molecule. The phrase “ING” shall also include all enantiomers and diastereomers thereof in all proportions. The phrase “KMN-159” shall likewise refer to the free acid, pharmaceutically acceptable salts, as well as its lower alkyl esters, in particular the isopropyl ester and all diastereomers in all proportions. Likewise, for all EP2 and EP4 and FP agents, it is specifically contemplated that their free acids, or free acid equivalents, salts, alkyl esters, monoalkyl amides, and NO-releasing esters are included.

### Buffers

Suitable buffers include, but are not limited to, acetic acid, citric acid, carbonic acid, phosphoric acid, boric acid, the pharmaceutically acceptable salts thereof, and tromethamine. The buffer may be present in an amount of from about 0.01% weight/volume to about 1% weight/volume or about 0.1% w/v to about 0.9% w/v, or about 0.3% w/v to about 0.8% w/v, or about 0.4% w/v to about 0.6% w/v. Suitably, the buffer may be present in at least 0.01% w/v, at least 0.05% w/v, at least 0.1% w/v, at least 0.3% w/v, or at least 0.5% w/v. Suitably, the buffer may be present in an amount of no more than 1.0% w/v, no more than 0.8% w/v, no more than 0.6% w/v, or no more than 0.5% w/v.

### Tonicity Agents

The tonicity, or osmolality, of the product can be adjusted to hypotonicity, isotonicity, or hypertonicity relative to normal tears by use of conventional materials known in the art. Tonicity agents are typically ionic compounds. Examples of tonicity agents include, but are not limited to, sodium chloride, potassium chloride, mannitol, dextrose, glycerine, and propylene glycol. Suitably, the tonicity agent may be present in an amount of from about 0.01% weight/volume to about 10% weight/volume or about 1% w/v to about 10% w/v, or about 2.5% w/v to about 7.5% w/v, or about 4% w/v to about 6% w/v. Suitably, the tonicity agent may be present in at least 0.01% w/v, at least 0.05% w/v, at least 1% w/v, at least 2.5% w/v, at least 4% w/v, or at least 5% w/v. Suitably, the tonicity agent may be present in an amount of no more than 10% w/v, no more than 7.5% w/v, no more than 6% w/v, or no more than 5% w/v.

### Other Components

In embodiments, the composition may also contain pH adjusting agents in an amount sufficient to adjust the pH of the composition to between about 4.5 to about 6.5. Suitably, the composition has a pH of about 4.5 to about 6.2 or about 4.5 to about 6.0. Suitable pH adjusting agents include, but are not limited to, sodium hydroxide.

In embodiments, the composition may also contain an emulsifying agent. Suitable emulsifying agents include, but are not limited to, polyoxyl 40 stearate, polyethoxylated castor oil, and combinations thereof, and other agents known to one skilled in the art.

Ophthalmic products are typically packaged in unit dose or multidose form. Preservatives may be present to prevent or inhibit microbial contamination. Suitable preservatives include, but are not limited to, benzalkonium chloride, thimerosal, chlorobutanol, methyl paraben, propyl paraben, phenylethyl alcohol, edtate disodium, boric acid, sorbic acid, or other agents known to one skilled in the art. If present, the preservative may be present in an amount of about 0.001% w/v to about 1.0% w/v, or about 0.01% w/v to about 1.0% w/v, or about 0.1% w/v to about 1.0% w/v. Suitably, the preservative may be present in at least 0.001% w/v, at least 0.005% w/v at least 0.01% w/v, at least 0.05% w/v, at least 0.1% w/v, or at least 0.5% w/v. Suitably, the preservative may be present in an amount of no more than 1.0% w/v, no more than 0.9% w/v, no more than 0.5% w/v, or no more than 0.1% w/v.

Other components commonly used in ophthalmic compositions, such as surfactants, comfort-enhancing agents such as menthol and WS-12 ((1R,2S,5R)—N-(4-methoxyphenyl)-5-methyl-2-propan-2-ylcyclohexane-1-carboxamide), solubilizing agents, antioxidants, and stabilizing agents, may also be present.

### Formulations and Methods of Use

The ophthalmic compositions of the present invention may be formulated in various dosage forms suitable for topical ophthalmic delivery, including solutions, suspensions, emulsions, and gels. The formulations may comprise carriers including but not limited to water, emulsifiers, or a gelling agent. The compositions are suitably aqueous.

The methods comprise topically applying to the affected eye(s) of the dog, cat or horse a beneficial amount of a composition according to the present invention. The frequency and amount of dosage can be readily determined by one of skill in the art based on various clinical factors. The methods typically comprise topical application of one or two drops once or twice a day.

In embodiments, the composition may comprise a single active pharmaceutical ingredient. In other embodiments, the composition may comprise a second active pharmaceutical ingredient. The second active pharmaceutical ingredient may be another EP2 or EP4 selective agent, or, especially for dogs, an FP-selective agent, such as latanoprost or travoprost. If present, the second active pharmaceutical ingredient may be present in an amount of about 0.001% w/v to about 1.0% w/v, or about 0.01% w/v to about 1.0% w/v, or about 0.1% w/v to about 1.0% w/v. Suitably, the second active pharmaceutical ingredient may be present in at least 0.001% w/v, at least 0.005% w/v at least 0.01% w/v, at least 0.05% w/v, at least 0.1% w/v, or at least 0.5% w/v, at least 5% w/v. Suitably, the second active pharmaceutical ingredient may be present in an amount of no more than 1.0% w/v, no more than 0.9% w/v, no more than 0.5% w/v, or no more than 0.1% w/v.

Other components commonly used in ophthalmic compositions, such as surfactants, comfort-enhancing agents, solubilizing agents, antioxidants, and stabilizing agents, may also be present.

### Alternative Formulations and Methods of Use

The ophthalmic compositions of the present invention may be formulated in various dosage forms suitable for topical ophthalmic delivery, including a carrier such as a biodegradable implant for injection. The biodegradable implant is formulated with standard bioerodible polymers and include one or more EP2, EP4 or FP agents. The compositions are suitably aqueous.

The present invention is directed to methods of treating glaucoma, ocular hypertension (OHT) and other ophthalmic diseases. As used herein, “treat” or “treating” as used herein refers to administering a regimen to the subject, e.g., the administration a compound or composition described herein, such that the disorder or at least one symptom of the disorder is healed, alleviated, relieved, altered, remedied, ameliorated, and/or improved. For example, after dosing, the Intraocular Pressure (IOP) of the animal decreases by at least about 10%, by at least about 20%, or by at least about 25% or more, and remains low for a period of time. Treating includes administering an amount effective to alleviate, relieve, alter, remedy, ameliorate, improve and/or affect the disorder or the symptoms of the disorder. The treatment may inhibit deterioration or worsening of a symptom of a disorder.

The methods comprise topically applying to the affected eye(s) of the dog, cat or horse a therapeutically effective amount of a composition according to the present invention. The frequency and amount of dosage can be readily determined by one of skill in the art based on various clinical factors. The methods typically comprise topical application of one or two or three drops administered once or twice a day.

### EXAMPLES

The present disclosure has multiple aspects, illustrated by the following non-limiting examples. In the various examples, the below materials and characterization techniques have been used.

### Example 1. Formulations with ING

Table 1 shows the composition of selected formulations. Formulations A, B and C are prepared by adding boric acid to D-mannitol with or without 0.015% benzalkonium chloride in a labeled 150-milliliter (mL) plastic storage container. ING, with or without 5% benzalkonium chloride stock solution, are then added and dissolved by stirring the solution for another 10 minutes. 100 milliliters (mL) of purified water are then added to bring the solution almost to 100%, and the pH was adjusted to approximately 6.0.

TABLE 1

| | Formulation (% weight/volume) | | |
|---|---|---|---|
| Ingredient | A | B | C |
| ING | 0.01 | 0.005 | 0.002 |
| Boric acid | 0.05 | 0.05 | 0.05 |
| D-mannitol | 4.7 | 4.7 | 4.7 |
| Benzalkonium chloride | — | 0.015 | 0.015 |
| Purified water | q.s. | q.s. | q.s. |
| pH | 5.8 | 5.8 | 6.0 |

### Example 2. Formulations Comprising Fixed-Dose Combinations of ING and an FP-Selective Agent

Table 2 shows the formulations of fixed-dose combination of ING and latanoprost. Formulations D and E are prepared by the same method as described above.

TABLE 2

| | Formulation (% | |
|---|---|---|
| | weight/volume) | |
| Ingredient | D | E |
| ING | 0.01 | 0.005 |
| Latanoprost | 0.005 | 0.005 |
| Boric acid | 0.05 | 0.05 |
| D-mannitol | 4.7 | 4.7 |
| Benzalkonium chloride | 0.015 | 0.02 |
| Purified water | q.s. | q.s. |
| pH | 5.8 | 5.5 |

### Example 3. Preservative-Free, Fixed-Dose Combination of ING and a FP-Selective Agent

Table 3 shows formulations comprising preservative-free, fixed-dose combinations of ING and travoprost. Formulations F, G and H are made by the technique as described above.

TABLE 3

| | Formulation (% weight/volume) | | |
|---|---|---|---|
| Ingredient | F | G | H |
| ING | 0.02 | 0.01 | 0.005 |
| travoprost | 0.005 | 0.005 | 0.005 |
| Boric acid | 0.05 | 0.05 | 0.05 |
| D-mannitol | 4.7 | 4.7 | 4.7 |
| Polyoxyl 40 stearate | 0.5 | — | — |
| (Myrj-52) | | | |
| Cremophor RH 40 | — | 0.25 | 0.5 |
| Benzalkonium chloride | — | — | — |
| Purified water | q.s. | q.s. | q.s. |
| pH | 5.5 | 5.5 | 5.5 |

Table 4 shows the composition comprising ING and netarsudil dimesylate. Formulation J, K, and L are prepared by adding boric acid, D-mannitol, and with or without 0.015% benzalkonium chloride in a labeled 150-milliliter (mL) plastic storage container. The netarsudil mesylate and ING, with or without 5% benzalkonium chloride stock solution, are then added and dissolved by stirring the solution for another 10 minutes. 100 milliliters (mL) of purified water are then added to bring the solution almost to 100%, and the pH is adjusted to approximately 5.0.

TABLE 4

| | Formulation (% weight/volume) | | |
|---|---|---|---|
| Ingredient | J | K | L |
| Netarsudil dimesylate | 0.03 | 0.015 | 0.015 |
| ING | 0.01 | 0.005 | 0.0015 |
| Boric acid | 0.05 | 0.05 | 0.05 |
| D-mannitol | 4.7 | 4.7 | 4.7 |
| Benzalkonium chloride | 0.015 | 0.015 | 0.008 |
| Purified water | q.s. | q.s. | q.s. |
| pH | 5.0 | 4.8 | 4.9 |

It is understood that the foregoing detailed description and accompanying examples are merely illustrative and are not to be taken as limitations upon the scope of the invention, which is defined solely by the appended claims and their equivalents.

1. An ophthalmic composition for dogs, cats and horses, comprising:
a selective EP2 or EP4 agent and
a carrier.

2. An ophthalmic composition for dogs, cats and horses, comprising:
ING or KMN-159; and
about 0.01% weight/volume of a tonicity agent.

3. An ophthalmic composition for dogs, cats and horses, comprising:
ING or KMN-159; and
about 0.0001% weight/volume to about 1.0% weight/volume of an FP agent or netarsudil; and
about 0.01% weight/volume to about 10% weight/volume of a tonicity agent.

4. The composition of claim 2, wherein the ING or KMN-159 is present as its isopropyl ester.

5. The composition of claim 1, wherein the pH is from about 4.5 to about 6.2.

6. The composition of claim 1, further comprising a preservative.

7. The composition of claim 6, wherein the preservative comprises benzalkonium chloride.

8. The composition of claim 1, further comprising an emulsifying agent.

9. The composition of claim 8, wherein the emulsifying agent comprises polyoxyl 40 or polyoxyl 35 castor oil, stearate, polyethoxylated castor oil, or a combination thereof.

10. The composition of claim 1, further comprising a second ophthalmic active compound.

11. The composition of claim 10, wherein the second ophthalmic active compound is an FP agent.

12. The composition of claim 11, wherein the FP agent is AR-102, latanoprost, travoprost, bimatoprost, latanoprostene bunod, or tafluprost.

13. The composition of claim 1, wherein the formulation is dosed to a dog, cat or horse once or twice a day.

14. The composition of claim 1 wherein the carrier is a bioerodible polymer.

15. A method for treating glaucoma, ocular hypertension (OHT) and other ophthalmic diseases in an animal in need thereof, the method comprising administering a therapeutically effective amount of a composition comprising a selective EP2 and/or EP4 agent as a free acid, pharmaceutically acceptable salts, its lower alkyl esters, or diastereomers thereof, and an excipient, diluent or carrier.

16. A method for treating glaucoma, ocular hypertension (OHT) and other ophthalmic diseases in an animal in need thereof, the method comprising administering a therapeutically effective amount of a composition comprising at least one active agent selected from: isopropyl N-(6-(((4-(1H-pyrazol-1-yl)benzyl) (3-pyridinylsulfonyl)amino)methyl)-2-pyridinyl)glycinate (ING) and (7-((R)-3,3-difluoro-5-((3S,4S,E)-3-hydroxy-4-methylnon-1-en-6-yn-1-yl)-2-oxopyrrolidin-1-yl) heptanoic acid (KMN-159) including its free acid, pharmaceutically acceptable salts, its lower alkyl esters, or diastereomers thereof; and a tonicity agent.

17. The method of claim 16, wherein the composition further comprises an FP agent selected from the group comprising AR-102, latanoprost, travoprost, bimatoprost, latanoprostene bunod, and tafluprost or netarsudil.

18. The method of claim 16, wherein the ING or KMN-159 is present in the composition as an isopropyl ester.

19. The method of claim 15, wherein the composition is formulated as a liquid composition, wherein the EP2 and/or EP4 agents are present in the composition in an amount ranging from about 0.0001% weight/volume to about 5% weight/volume.

20. The method of claim 15, wherein the composition further comprises at least one of a kinase inhibitor and a beta blocker.

21. The method of claim 19, wherein the composition is administered to the eye of an animal selected from a dog, a cat and a horse.

22. The method of claim 21, wherein the composition is formulated for topical application.

23. The method of claim 15, wherein administration of the composition to a dog, a cat and a horse, results in the reduction of IOP in the treated eye of said dog, cat and horse, by at least about 10%, by at least about 20%, or by at least about 25% or more, after the first, or multiple administrations during the course of treatment.
